(19) United States
(12) Patent Application Publication
(10) Pub. No.: US 2006/0292195 A1
(43) Pub. Date: Dec. 28, 2006

(54) Tooth drops

(76) Inventor: Herbert Barkley Woosley, Brookneal, VA (US)

Correspondence Address: MICHAEL RIES, 1747A VERMONT DRIVE, ELK GROVE, IL, 60007, US

(21) Appl. No.: 11/472,037

(22) Filed: Jun. 21, 2006

Related U.S. Application Data
(60) Provisional application No. 60/692,783, filed on Jun. 22, 2005.

(51) Int. Cl. A61K 8/02; A61K 9/48

(52) U.S. Cl. 424/401; 424/451

(57) ABSTRACT

Tooth cleaning drops with an outer shell defining an inner chamber filled with a tooth cleaning substance. The outer shell may be made of the same material as the tooth cleaning substance. The drops are placed in a user's mouth and punctured to release the tooth cleaning substance held inside and may be used with or without a tooth brush. The apparatus may have a two-part housing defining spaces for forming the drops. The apparatus and drops may be heated or cooled. The drops may be sealed or sprayed. The drops eliminate the need for messy tooth paste tubes and make brushing easier and more convenient.

This application claims the benefit of U.S. Provisional Patent Application Ser. No. 60/692,783, filed Jun. 22, 2005.

### TECHNICAL FIELD & BACKGROUND

The present invention generally relates to the field of oral hygiene. More specifically, the present invention relates to tooth cleaning drops, methods of using the drops, an apparatus for manufacturing the drops, and a method for manufacturing the drops.

There is a constant search in the oral hygiene industry to find new ways to improve the process of cleaning teeth with tooth cleaning materials. A well-known means of brushing teeth is the use of a tooth brush with tooth paste. Other products on the market today include disposable textured tooth wipes; however, such products require manipulation with the fingers, can be messy to use and produce waste.

The present invention delivers a tooth cleaning product in an easy-to-use capsule without the need for messy tooth paste tubes. Also, the present invention is typically used with a tooth brush however, the present invention may optionally be used without a tooth brush. The drops may be used alone when a tooth brush is not available.

### BRIEF DESCRIPTION OF THE DRAWINGS

The present invention will be described by way of exemplary embodiments, but not limitations, illustrated in the accompanying drawings in which like references denote similar elements, and in which:

FIG. 1 is a perspective view of an embodiment of an apparatus for manufacturing tooth cleaning drops in an open position.

FIG. 2 is a detail perspective view of a dispensing nozzle within an opening within the apparatus of FIG. 1.

FIG. 3 is a side sectional view through the apparatus showing one step in a method of manufacturing tooth cleaning drops.

FIG. 4 is a side sectional view through the apparatus showing a step in a method of manufacturing tooth cleaning drops subsequent to FIG. 3.

FIG. 5 is a side sectional view through the apparatus showing a step in a method of manufacturing tooth cleaning drops subsequent to FIG. 4.

FIG. 6 is a side sectional view of a tooth cleaning drop between the step shown in FIG. 3 and the step shown in FIG. 4.

FIG. 7 is a side sectional view of a tooth cleaning drop after the step shown in FIG. 5.

FIG. 8 is a detail partial sectional view of the tooth cleaning drop shown in FIG. 7.

### DETAILED DESCRIPTION OF ILLUSTRATIVE EMBODIMENTS

Various aspects of the illustrative embodiments will be described using terms commonly employed by those skilled in the art to convey the substance of their work to others skilled in the art. However, it will be apparent to those skilled in the art that the present invention may be practiced with only some of the described aspects. For purposes of explanation, specific numbers, materials and configurations are set forth in order to provide a thorough understanding of the illustrative embodiments. However, it will be apparent to one skilled in the art that the present invention may be practiced without the specific details. In other instances, well-known features are omitted or simplified in order not to obscure the illustrative embodiments.

Various operations will be described as multiple discrete operations, in turn, in a manner that is most helpful in understanding the present invention, however, the order of description should not be construed as to imply that these operations are necessarily order dependent. In particular, these operations need not be performed in the order of presentation. The phrase in one embodiment is used repeatedly. The phrase generally does not refer to the same embodiment, however, it may. The terms comprising, having and including are synonymous, unless the context dictates otherwise.

The present invention is directed to tooth cleaning drops, methods of using the drops, an apparatus for manufacturing the drops, and a method for manufacturing the drops. The drops may comprise a tooth cleaning material such as toothpaste or tooth gel encapsulated in a sphere of denser tooth cleaning material. The drops replace the need for messy tooth paste tubes or bottles. The drops are provided for the purpose of introducing tooth cleaning substances to the mouth of an animal having teeth. Although the present invention is generally intended for use by humans, it is to be understood that the present invention may be provided for any animal having teeth that require cleaning, particularly pets such as dogs or cats.

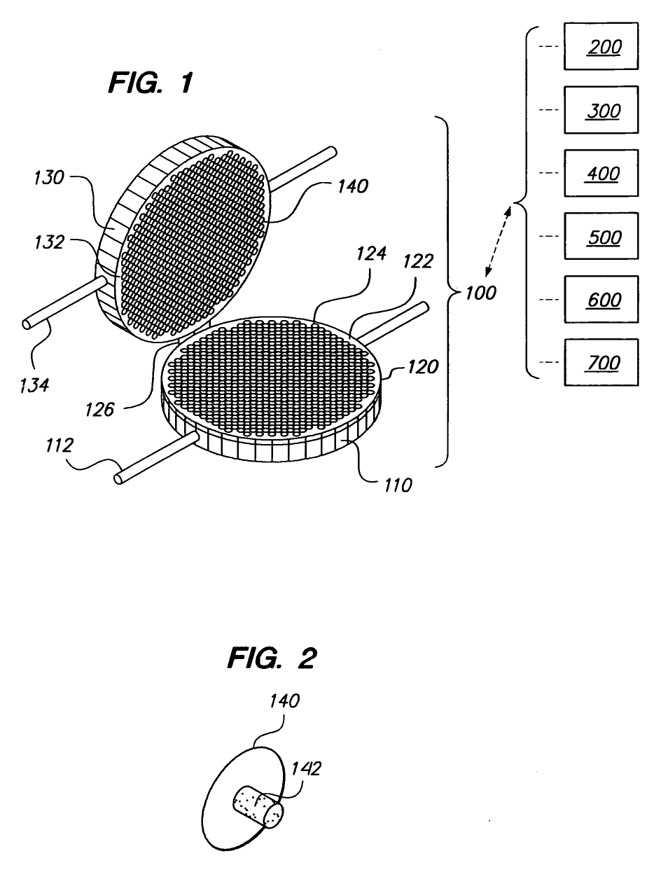

Referring now to FIG. 1, an apparatus 100 for manufacturing a cleaning device or tooth cleaning drop 10 (shown in FIG. 4) for teeth is shown. The apparatus 100 is a cost-effective means for producing the cleaning devices 10. The apparatus 100 may comprise a base 110 and an upper plate 130 connected via hinge 126. The base 110 may comprise a detachable ring 120. The ring 120 may comprise an upper face 122, and a plurality of openings 124 may be disposed in the upper face 122 of the ring 120. The upper plate 130 may comprise a lower face 132, and a plurality of openings 140 may be disposed in the lower face 132 of the upper plate 130. The openings 124, 140 may be formed using any suitable means of manufacture such as milling or casting. In an embodiment where the detachable ring 120 is omitted, it is understood that the base 110 itself may be provided with an upper face 122 and a plurality of openings 124.

In FIG. 1, the openings 124, 140 may have a generally semi-spherical shape and may form a generally spherical shape when the upper plate 130, and base/ring 110/120 assembly are brought together into a closed position (see, for example, FIG. 4). Although the present invention is described showing semi-spherical openings 124, 140 that join to define a spherical chamber, which, in turn, is used for forming spherical cleaning devices 10, it is to be understood that any suitable shape may be provided for the openings 124, 140 and for the cleaning devices 10 produced in the openings 124, 140.

For example, the cleaning devices 10 may be provided in a generally cubical shape, a generally cylindrical shape or an irregular shape. The term “generally” is used to denote the fact that, in reality, no shape is perfectly spherical, cubical or cylindrical. For example, in the case of a generally spherical cleaning device, the diameter may not be uniform throughout the cleaning device. Also, in the case of a cubical cleaning device, the cleaning device may have rounded edges.

In the case of a generally spherical cleaning device 10, the size should be sufficient to hold enough cleaning material to be suitable for one cleaning operation. Specifically, the size of the cleaning device 10 may be adapted to hold enough cleaning material to be suitable for one cleaning operation for the use of one adult or one child. The size may be varied for a particular age of the user. For example, various sizes may be provided to hold an amount of cleaning material that is appropriate for a baby, for a child that is two to three years old, for adolescents and for teenagers and adults. In one embodiment of a cleaning device for an adult, the size of the cleaning device 10 may be about 16.0 mm in diameter. For use with animals such as pets, various sizes may be provided to hold an amount of cleaning material that is appropriate for a small, medium or large dog or cat.

In FIG. 1, the apparatus 100 may be provided with a means for rotating the apparatus 100. For example, such means may comprise rotating rods 112 attached to the base 110 and rotating rods 134 attached to the upper plate 130. When the base 110 and upper plate 130 are closed, the rods 112 and 134 may be used to rotate the apparatus 100.

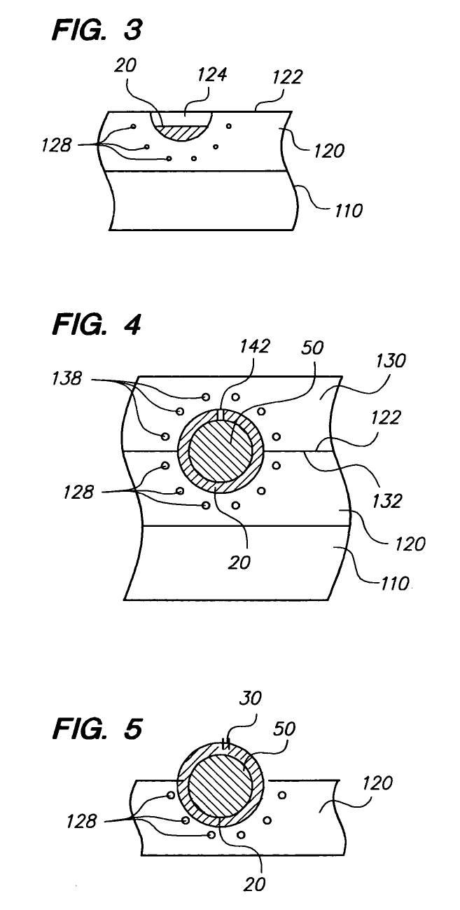

The apparatus 100 may be provided with a means for introducing a gas or fluid into the apparatus 100 so that the gas or fluid passes close to the openings 124 and 140 formed in the ring 120 and upper plate 130, respectively. For example, as shown in FIGS. 3-5, the ring 120 may be provided with a plurality of through holes 128 located close to the surface of the opening 124, and the upper plate 130 may be provided with a plurality of through holes 138 located close to the surface of the opening 140. The means for introducing a gas or fluid may be connected to the apparatus 100 in any suitable manner. For example, the means for introducing a gas or fluid may be connected through openings in the rods 112 and 134.

Although six circular openings are shown in FIGS. 3-5 in each of the ring 120 and the upper plate 130, any suitable shape or number of openings may be provided. The distance between through holes 128, 138 and their respective openings 124, 140 should be sufficient to permit efficient transfer of energy to or from the gas or fluid contained in the holes 128, 138. That is, the gas or fluid contained in the holes 128, 138 may be used to heat or cool the openings 124, 140 and any material inside the openings 124, 140. Alternately, the openings 124, 140 may be distributed throughout the ring 120 and the upper plate 130 so that the entire mass of the ring 120 and upper plate 130 may be heated or cooled as desired.

The apparatus 100 may comprise a multi-port nozzle 142. In one embodiment, as shown in FIG. 2, the nozzle 142 may be provided inside each of the plurality of openings 140 in upper plate 130. The nozzle 142 may be adapted to inject one or more types of material into the opening formed by the combined openings 124, 140. One or more nozzles 142 may be used for each opening. When the nozzle 142 is used as a means 500 for introducing a second material (described below), the multi-port nozzle 142 promotes an even dispersal of material without compromising the wall thickness of the outer shell 20.

Portions of the apparatus in contact with the cleaning devices may be formed from materials that are easily heated and cooled and that permit the transfer of energy to and from the cleaning devices 10 formed in the apparatus 100. For example, the base 110, ring 120 and upper plate 120 may comprise a material selected from the group consisting of copper, beryllium, magnesium and aluminum. Also, a mixture of any of these materials may be used, for example a mixture of copper and beryllium. Other suitable materials that perform this function may be used either alone or in combination with each other or the materials listed above.

The apparatus 100 may comprise a means 200 for heating the apparatus 100. For example, hot gas or liquid may be injected into the apparatus using through holes 128 and 138. In another embodiment, a heating unit may be placed in contact with the apparatus 100. In yet another embodiment, the apparatus 100 may be placed in a heated environment. An example of how a heating means 200 may be used with the apparatus 100 will be described in greater detail below.

The apparatus 100 may comprise a means 300 for cooling the apparatus 100. For example, cold gas or liquid may be injected into the apparatus using through holes 128 and 138. In another embodiment, a cooling unit may be placed in contact with the apparatus 100. In yet another embodiment, the apparatus 100 may be placed in a cooled environment. An example of how a cooling means 300 may be used with the apparatus 100 will be described in greater detail below.

The apparatus 100 may comprise a means 400 for introducing a first material into the apparatus 100. The first material is used to form the outer shell 20 of the cleaning device 10. The first material may be formed from the same material as the second material (see discussion below), which is used to form the contents 50 of the cleaning device 10. For example, the first material may be a tooth paste or gel with the addition of a material that increases the density or consistency of the tooth paste or gel. The material for increasing the density or consistency may be starch or baking soda in any suitable concentration to form the outer shell 20. The first material may be introduced into the apparatus 100 using any suitable method including dropping the first material from above or injecting the material through a nozzle such as nozzle 142. An example of how a first material introducing means 400 may be used with the apparatus 100 will be described in greater detail below.

The apparatus 100 may comprise a means 500 for introducing a second material into the apparatus 100. The second material is used to form the contents 50 of the cleaning device 10. For example, the second material may be a tooth paste or gel. For example, tooth paste or gel may comprise materials in the following proportions: about 75% humectant and water, about 20% abrasive, about 2% foaming and flavoring agents, about 2% pH buffers, less than about 1.5% binders and opacifiers, and about 0.24% fluoride. Any suitable variation of these materials may be used to form the second material. Certain materials may be omitted, and additional materials may be added to form the second material. The second material may be introduced into the apparatus 100 using any suitable method. For example, the second material may be injected through a nozzle such as nozzle 142. An example of how a second material introducing means 500 may be used with the apparatus 100 will be described in greater detail below.

The apparatus 100 may comprise a means 600 for sealing the cleaning device 10. The sealing means 600 may be a heater, such as a heated belt. An example of how a sealing means 600 may be used with the apparatus 100 will be described in greater detail below.

The apparatus 100 may comprise a means 700 for spraying a material on the cleaning device 10. The spraying means 700 may be a spray system for spraying a food varnish or lacquer on the cleaning devices 10 after formation. The food varnish or lacquer may be a material that enhances the color of the cleaning devices 10. The food varnish or lacquer may be a material that prevents the cleaning devices 10 from sticking together during packaging and subsequent transit. An example of how a spraying means 700 may be used with the apparatus 100 will be described in greater detail below.

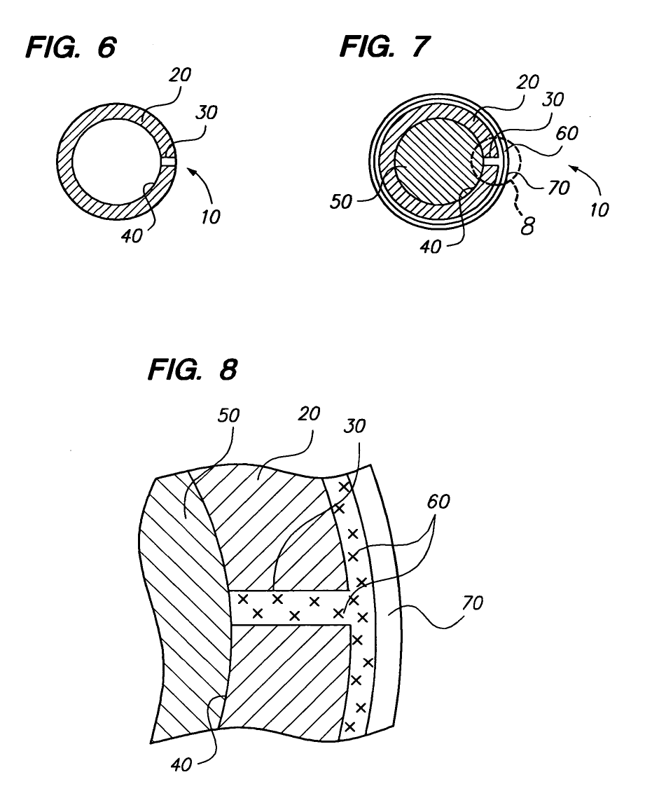

In one embodiment of the present invention, a device 10 comprises a capsule capable of being placed in a human mouth, and an oral hygiene substance 50 (or oral hygiene fluoridation substance) inside the capsule that leaves the inside of the capsule and floods the mouth and teeth of a user to allow a person to perform oral hygiene by brushing their teeth with a tooth brush and the oral hygiene substance 50. Also, the present invention is directed to a method of using the device 10, where the method may comprise placing a device 10 in a human mouth, releasing an oral hygiene substance 50 from an inside of the device 10, flooding the mouth and teeth of a person with the oral hygiene substance 50, and performing oral hygiene by brushing the person's teeth with a tooth brush and the oral hygiene substance 50. The device 10 may be shaped substantially like a sphere (see, for example, FIGS. 6 and 7). The oral hygiene substance 50 may be tooth paste or a tooth gel. The device 10 may be water soluble. The capsule may take the form of an outer shell 20 (see, for example, FIGS. 6-8). The capsule may have an abrasive to aid in oral hygiene. A person using the device 10 may bite down on the capsule to release the oral hygiene substance 50. In another embodiment, a person may perform oral hygiene by rubbing the oral hygiene substance 50 from the device 10 on the teeth with their tongue or any other appropriate material instead of using a tooth brush.

In another embodiment of the present invention, a cleaning device 10 for teeth comprises an outer shell 20 surrounding an inner chamber 40, a tooth cleaning substance 50 provided inside the inner chamber 40, where the tooth cleaning substance 50 comprises toothpaste or tooth gel, and where the outer shell 20 comprises a mixture of the toothpaste or tooth gel and a means for increasing a density of the toothpaste or tooth gel. The means for increasing the density of the toothpaste or tooth gel may be a starch or a baking soda. The cleaning device 10 may further comprise an opening 30 in the outer shell 20, a seal 60 blocking the opening 30, where the seal 60 comprises a melted portion of the outer shell 20. The cleaning device 10 may further comprise a food varnish or lacquer coating 70 on the outer shell 20. The seal 60 may cover a portion of the outer shell 20 or the entire outer shell 20. As such, the coating 70 would cover the entire device 10 including the outer shell 20 and the seal 60. The cleaning device 10 may be provided in a size adapted to hold an amount of toothpaste or tooth gel suitable for a single use by an adult or child. The cleaning device 10 may have a generally spherical shape and may have an outside diameter of about 16 mm.

The present invention is also directed to an apparatus 100 for manufacturing a cleaning device 10 for teeth. The apparatus 100 may comprise a unit, the unit comprising a means for forming the cleaning device, for example, base 110, ring 120, openings 124 in upper face 122, upper plate 130 and openings 140 in lower face 132 may form the unit.

A cleaning device 10 for teeth may be formed by any suitable method. For example, a method for manufacturing a cleaning device 10 for teeth, may comprise introducing a first material 20 into an opening 124 formed in a base 110 and/or ring 122 of an apparatus 100, when the apparatus 100 is in an open position (FIG. 1). FIG. 2 illustrates a condition after the first material 20 has been introduced into the opening 124. The first material 20 may be introduced using any suitable means 400 for introducing the first material 20. For example, the first material 20 may be dropped from a dispenser located above the apparatus 100. In another embodiment, the apparatus 100 may be in a closed position, and the first material 20 may be injected through a nozzle 142 formed in the second opening 140 in the upper plate 130.

After the first material 20 has been introduced into the apparatus 100, if the apparatus 100 has not already been closed, it may be closed. After the apparatus 100 is closed, the apparatus 100 forms a chamber defined by the openings 124 and 140. The apparatus 100 may be rotated thus uniformly dispersing the first material 20 inside of the chamber formed by the combination of openings 124 and 140. The apparatus 100 may be rotated at a speed sufficient so that centrifugal force promotes uniform dispersal of the first material 20.

The first material 20 is cooled using a suitable means 300 for cooling. The cooling step may take place during the rotation step and/or after the rotation step. The first material 20 is thus cooled into an outer shell (FIG. 6) that has an opening 30 defined by nozzle 142. If intermediate heating is desired, it may be heated using a suitable means 200 for heating.

The second material 50 may be introduced into the apparatus 100 using any suitable means 500 for introducing a second material 50. FIG. 4 illustrates an example of the device 10 after introduction of the second material 50. If an excess amount of the second material 50 is injected into the chamber, the waste may be collected and added back to the source.

The apparatus 100 may be opened and a detachable portion 120 of the apparatus 100 may be lifted away from the main body of the apparatus 100. The partially formed devices 10 may be sealed using any suitable means 600 for sealing. For example, the devices 10 may be removed from the detachable portion 120 and placed on a declined heating belt. If the devices 10 are generally spherical, the devices 10 will be capable of rolling down a declined heating belt. As the devices 10 roll down the belt, the heat from the belt melts the outer shell 20, and the openings 30 in the devices 10 are thus sealed with melted material from the outer shell 20. Although FIGS. 6-8 indicate that the entire outer shell 20 is melted in order to form a seal 60, it is understood that the openings 30 in the outer shell 20 may be spot sealed thus forming a cap in the opening 30. For example, instead of removing the devices 10 from the detachable portion 120, since the openings 30 may be uniformly provided on the top of each device 10, the devices 10 may be spot sealed from above with a heating device or by dropping a sealing material onto the devices 10. Alternately, the openings 30 in the devices 10 may be sealed by heating the devices 10 with a means 200 for heating thus melting the outer shell 20 and forming a seal 60 comprising melted outer shell material (see, for example, FIG. 8).

The devices 10 may be sprayed with a third material 70 such as a food varnish or lacquer coating. After sealing and spraying the devices 10, each of the devices 10 might appear as shown in FIG. 7.

While the present invention has been related in terms of the foregoing embodiments, those skilled in the art will recognize that the invention is not limited to the embodiments depicted. The present invention can be practiced with modification and alteration within the spirit and scope of the appended claims. Thus, the description is to be regarded as illustrative instead of restrictive on the present invention.

What is claimed is:

1. A device comprising:
a capsule capable of being placed in a human mouth; and
an oral hygiene substance inside the capsule that leaves an inside of the capsule and floods the mouth and teeth to allow a person to perform oral hygiene by applying the oral hygiene substance to the teeth and mouth.

2. The device of claim 1 wherein the capsule is a selected one of shaped substantially like a sphere, water soluble and has an outer shell having an abrasive to aid in oral hygiene.

3. The device of claim 1 wherein the oral hygiene substance is a selected one of a tooth paste and a tooth gel.

4. The device of claim 1 wherein the applying the oral hygiene substance to the teeth is a selected one of brushing the teeth and using a material to move the oral hygiene substance on the teeth and around the mouth.

5. The device of claim 1, wherein the person bites down on the capsule to release the oral hygiene substance.

6. A method comprising:
placing a capsule in a human mouth,
releasing an oral hygiene substance from an inside of the capsule;
flooding the mouth and teeth of a person with the oral hygiene substance; and
performing oral hygiene by a selected one of brushing teeth of the person with a tooth brush along with the oral hygiene substance and moving the oral hygiene substance on the teeth with a material.

7. The method of claim 6, wherein the capsule is a selected one of shaped substantially like a sphere, water soluble and has an outer shell having an abrasive to aid in oral hygiene the oral hygiene substance is a selected one of a tooth paste and tooth gel, the person bites down on the capsule to release the oral hygiene substance.

8. A device comprising:
a capsule capable of being placed in a human mouth; and
an oral hygiene fluoridation substance inside the capsule that leaves the inside of the capsule and floods the mouth and teeth to allow a person to perform oral hygiene with the oral hygiene substance.

9. The device of claim 15 wherein the person performs oral hygiene by rubbing the oral hygiene substance on their teeth with their tongue.

10. The device of claim 15 wherein the person performs oral hygiene by rubbing the oral hygiene substance on their teeth with a tooth brush.

11. A cleaning device for teeth comprising:
an outer shell surrounding an inner chamber,
a tooth cleaning substance provided inside said inner chamber,
wherein said tooth cleaning substance comprises a selected one of toothpaste and tooth gel,
wherein said outer shell comprises a mixture of a selected one of the toothpaste and tooth gel and a means to increase a density of the selected one of the toothpaste and tooth gel.

12. The cleaning device of claim 11, wherein said means for increasing said density of the selected one of the toothpaste and tooth gel is a selected one of a starch and a baking soda.

13. The cleaning device of claim 11, wherein said cleaning device comprising:
an opening in said outer shell,
a seal blocking said opening, wherein the seal comprises a melted portion of the outer shell.

14. The cleaning device of claim 11, wherein said cleaning device comprising:
a selected one of a food varnish and lacquer coating on the outer shell.

15. The cleaning device of claim 11, wherein the cleaning device is provided in a size adapted to hold an amount of a selected one of the toothpaste and tooth gel suitable for a single use by various ages and amount needs of people.

16. The cleaning device of claim 11, wherein the cleaning device has a generally spherical shape and has an outside diameter of about 16 mm.

17. An apparatus for manufacturing a cleaning device for teeth, the apparatus comprising:
a unit comprising a means for forming the cleaning device;
a means for heating the unit;
a means for introducing a first material into the unit;
a means for rotating the unit;
a means for cooling the unit; and
a means for introducing a second material into the unit.

18. The apparatus of claim 17, wherein the unit comprises a detachable means, the detachable means comprising a portion of the means for forming the cleaning device.

19. The apparatus of claim 17, wherein the apparatus comprises a means for sealing the cleaning device.

20. The apparatus of claim 17, wherein the apparatus comprises a means for spraying a material on the cleaning device.

21. The apparatus of claim 17, wherein the unit comprises a multi-port nozzle.

22. The apparatus of claim 17, wherein the unit comprises a two-part structure, and the apparatus comprises a means for opening and closing the two-part structure of the unit.

23. The apparatus of claim 17, wherein the unit comprises a material selected from the group consisting of copper, beryllium, magnesium and aluminum.

24. A method comprising:
introducing a first material into a means for forming the cleaning device;
rotating the unit thus uniformly dispersing the first material inside of the means for forming the cleaning device;
cooling the first material;
introducing a second material into the unit;
sealing the cleaning device; and
spraying the cleaning device with a third material.
